(19) United States
(12) Patent Application Publication
(10) Pub. No.: US 2026/0057977 A1
(43) Pub. Date: Feb. 26, 2026

(54) Method for Predicting Communication Strategy With Patient and Server Computer Thereof

(71) Applicants: Kura Care, INC., San Diego, CA (US); KURA Med Inc., Taipei City (TW)

(72) Inventors: Ming-Tse Tsai, Poway, CA (US); Chih-Wei Chiu, Kaohsiung City (TW); Yu-Hsiu Kao, New Taipei City (TW); Kai-Chieh Yang, Taoyuan City (TW)

(21) Appl. No.: 19/307,228

(22) Filed: Aug. 22, 2025

Related U.S. Application Data
(60) Provisional application No. 63/687,011, filed on Aug. 26, 2024.

(51) Int. Cl. G16H 10/60; G16H 80/00

(52) U.S. Cl. CPC G16H 10/60; G16H 80/00

(57) ABSTRACT

A method for predicting a communication strategy with patient, comprising: receiving a patient's demographic data; finding a model persona in a persona database based on the patient's demographic data; generating one or more first tasks based on a first communication strategy which meets attribute data of the model persona in the persona database; sending sequentially the one or more first tasks based on the first communication strategy to a client computer of the patient via a network; receiving sequentially one or more first responses with respect to the one or more first tasks from the client computer; and recording a first interaction history including the one or more first tasks and the corresponding one or more first responses in the persona database.

### CROSS REFERENCE TO RELATED PATENT APPLICATION

This patent application is based on a provisional patent application No. 63/687,011 filed on Aug. 26, 2024.

### FIELD OF THE INVENTION

The present invention relates to network system, and more particularly, to a network system for predicting a communication strategy with patient based on persona database.

### BACKGROUND OF THE INVENTION

There are different kinds of patients who have different preferred times to communicate with and different capacity of engagement in a single encounter. It is time consuming for a clinic and a new patient to find out the right time and the right capacity of engagement. Also the preferred time of communication and the capacity of engagement for the patient might change along with the time.

Thus, there exists a need to provide a mechanism to suggest a time of communication and a capacity of engagement for a new patient by finding a comparable persona in patients' database.

### SUMMARY OF THE INVENTION

According to an embodiment of the present application, a method for predicting a communication strategy with patient is provided. The method for predicting a communication strategy with patient, comprising: receiving a patient's demographic data; finding a model persona in a persona database based on the patient's demographic data; generating one or more first tasks based on a first communication strategy which meets attribute data of the model persona in the persona database; sending sequentially the one or more first tasks based on the first communication strategy to a client computer of the patient via a network; receiving sequentially one or more first responses with respect to the one or more first tasks from the client computer; and recording a first interaction history including the one or more first tasks and the corresponding one or more first responses in the persona database.

According to an embodiment of the present application, a server computer, comprising: a networking device configured for connecting with a network; and a processor configured for executing instructions stored in non- volatile memory to realize the abovementioned method for predicting communication strategy with patient.

With the presented method and the server computer, a communication strategy for a new patient or a previously engaged patient with changed demographic data can be suggested quickly by finding a comparable persona in a persona database. And one or a combination of machine learning models may be used to suggest revised communication strategy based on several rounds of interactions between the patient and the network system. The attribute data of patients can be updated to the persona database to broaden the comparison basis. Moreover, interaction histories can be also used as a training set to train the one or a combination of machine learning models to improve the suggestions of communication strategies.

### BRIEF DESCRIPTION OF THE DRAWINGS

The advantages and spirit related to the present invention can be further understood via the following detailed description and drawings.

FIG. 1 illustrates a block diagram of a network system 100 for predicting communication strategy with patient.

FIG. 2 depicts a software architecture 200 implemented by a server for predicting a communication strategy based on demographic data in a persona database in accordance with an embodiment of the present application.

FIG. 3 illustrates a database schema 300 of the proposed persona database in accordance with an embodiment of the present application.

FIG. 4 depicts a block diagram of a computer 400 in accordance with an embodiment of the present application.

FIG. 5 shows a flowchart diagram of a method 500 for predicting communication strategy based on demographic data in a persona database in accordance with an embodiment of the present application.

FIGS. 6A through 6D depict parts of contents in a persona database in accordance with an embodiment of the present application.

### DETAILED DESCRIPTION OF THE PREFERRED EMBODIMENT

Some embodiments of the present application are described in detail below. However, in addition to the description given below, the present invention can be applicable to other embodiments, and the scope of the present invention is not limited by such rather by the scope of the claims. Moreover, for better understanding and clarity of the description, some components in the drawings may not necessary be drawn to scale, in which some may be exaggerated related to others, and irrelevant. If no relation of two steps is described, their execution order is not bound by the sequence as shown in the flowchart diagram.

One of the perspectives of the present application is to provide a network system for predicting communication strategy, including but not limiting to time, duration, topic, and capacity, with patient based on records in a persona database. The persona database in a network system collects different persona records, including two parts. The first part of the persona record comprises of persona demographic data and the second part of the persona record comprises persona attribute data.

The persona demographic data may comprise any combination of following: nickname, age, gender, social determinant of health (SDOH), working hours per week or per month, comorbidity, medication, symptoms, motivation, conversation tonicity, and/or special time event of a patient. The special time event may include but not limit to reset reminder time (e.g., 22:15 pm) and a time of home arriving (e.g., 7:15 pm). The persona attribute data may comprise any combination of following: preferred communication time, preferred communication duration, preferred communication topic, and capacity of tasks per day.

When a new patient or a patient with changed demographic data is received, a communication strategy may be searched and/or revised to meet the patient's needs. Therefore, in one embodiment, the proposed network system may view the patient with changed demographic data as a new record in the persona database. Thus, please be aware that a single patient may be related to one or more persona records in the database.

After receiving a new patient or a previously engaged patient with changed demographic data, the records in the persona database can be compared with the new patient or the patient with changed demographic data to find a model persona in the persona database. In one embodiment, the comparisons may be based on the demographic data of patients. The comparisons may be done in two ways, through deterministic algorithms or through machine learning models.

Since there are many columns in the demographic data in the persona database, in a first example of comparison algorithm, the model persona may be chosen because the record of the model persona has the most columns identical to the new patient. In a second example, differences corresponding to each column in the demographic data between existing records and the new patient are calculated. The record with the smallest sum of differences may be chosen as the model persona. In a variant of the second example, the differences may be weighted, respectively.

In the way of machine learning, one or a combination of machine learning models may be employed to find the model persona. The machine learning model may be trained based on the demographic data of the persona database. Alternatively, the machine learning model may be trained based on statistics demographic data provided by other social databases.

Some constraints or pre-requisites may be set up in finding the model persona. For example, a constraint including that the gender of the new patient and the model persona has to be the same may be required in the comparisons. Another constraint may require that the difference of ages is less than 3 years. Person having ordinary skill in the art can understand that the present application does not limit how to find the model persona closet to the new patient according to the demographic data.

Once the model persona is found, the persona attribute data corresponding to the model persona can be retrieved from the persona database as initial suggestions to the new patient. The suggested persona attribute data may comprise a communication strategy including one or any combination of following: preferred communication time, preferred communication duration, preferred communication topic, and capacity of tasks per day.

In one embodiment, an interaction history with the patient may be further recorded in order to provide more inputs to a prediction module. For example, the interaction history may include how long does it take for the patient to complete a task or a survey (i.e., related to the capacity of tasks), the patient's perspective about his own health issue (i.e., related to the preferred communication topic), and the time duration of the interaction (i.e., related to the preferred communication duration.)

The prediction module can use the suggested attribute data retrieved from the persona database and the optional interaction history inputs to predict the attribute data of the patient. Based on the predicted attribute data of communication strategy, the network system may communicate with the patient accordingly. In case the patient is satisfied with the predicted attribute data of communication strategy, this predicted attribute data can be updated to the persona database with corresponding demographic data of the patient by an update module.

In an embodiment, the interaction history may be analyzed to get the patient's tonicity preference. The analysis of tonicity preference may be performed by one or a combination of LLMs (large language models) based on the patient's responses. The analyzed tonicity preference may be recorded in the patient's demographic data and/or the attribute data in the persona database. Therefore, the system can communicate with the patient with the preferred tonicity next time. Following table gives examples of two different conversation manners/tonicities corresponding to the same context.

| context | “joking” manner | “coaching” manner |
|---|---|---|
| A friend messed up a | Well, at least you | You did well overall, |
| presentation | didn't set the | but next time try to slow |
| | projector on fire! | down and emphasize |
| | That was memorable. | your key points. You've |
| | | got this. |
| Teaching someone | If you hit one more | You're doing great. Just |
| how to drive | cone, I'm calling | remember to check your |
| | NASCAR to recruit | mirrors before changing |
| | you. | lanes. |
| A teammate forgets to | You forgot again? | It happens-let's set a |
| submit a report | Should we tattoo the | reminder next time so it |
| | deadline somewhere? | doesn't slip through. |

In some examples, the communication between the proposed system and the patient may be supported and provided by LLM with the context information and/or the tonicity preference as the prompts to the LLM. The answers of the patient in the interaction history may be also extracted by the LLM. Person having ordinary skill in the art can understand that the current LLMs are capable of the analysis of tonicity and generating outputs according to the tonicity requirements stated in the input prompt.

If it takes several rounds of communications to have the patient satisfied with the latest communication strategy, the latest communication strategy may be updated to the persona database with corresponding attribute data of the patient by the update module. Besides, the several rounds of communications may be further recorded as a part of training data. Another one or a combination of machine learning models can be trained according to the training data of rounds of communications. In an alternative embodiment, the prediction module may take advantage of the one or a combination of machine learning models for adjusting the predicted attribute data.

Please refer to FIG. 1, which illustrates a block diagram of a network system 100 for predicting communication strategy with patient in accordance with an embodiment of the present application. The network system 100 may comprise a server 110, a network 120, a clinic computer 130 for a clinic clerk, a client computer 140 for a patient and a DBMS server 112. Besides, the network system 100 may further comprise an optional AI (Artificial Intelligence) server 114, an optional message server 150.

The network 120 is configured for carrying data exchanges between the server 110 and the sensing device 130 and data exchanges between the server 110 and the client computer 140. For example, the network 120 may comprise access networks such as WiFi/IEEE 802.11 networks, 3G/4G/5G/6G compliant networks, IEEE 802.3 networks, PSTN networks, optical fiber networks, and/or xDSL networks. The server 110, the sensing device 130, the client computer 140, the optional AI server 112, and/or the message server 150 may be configured to connect to the access networks. Moreover, the network 120 may also comprise backbone networks, e.g., telecommunication networks, for connecting the access networks.

The exchange of information or messages between the server 110 and the clinic computer or the client computer 140 may be pass through the message server 150. The message server 150 may implement one or more public message service for the server 110 and the sensing device 130. For example, the message server 150 may be an electronic mail server, an instant message server (like Line, WhatsApp, Skype, Facetime etc.,) or a message queue server. One of the functions provided by the message server 150 is asynchronous transmission. It means that the message can be temporarily stored in the message server 150 and be resent by the message server 150 until it is safely received by the server 110. Another one of the functions provided by the message server 150 is encrypted transmission to guarantee the safety of the transmission. However, the message server 150 is optional in the embodiment of the present application.

As a variant of the message server 150, the SMS/MMS server 152 is able to transmit and receive short messages or multimedia messages to the clinic computer 130 and the client computer 140 from the server 110 and vice versa. The SMS/MMS server 152 may provide a scheduling function to the server 110. It means that the server 110 can upload the information and set the timing of transmitting the information in advance. Thus, when the preset time comes, the SMS/MMS server 152 can deliver the information which is packed in the short message or the multimedia message to the clinic computer 130 or the client computer 140 via a telecommunication network which may be included in the network 120. In addition, the SMS/MMS server 152 can receive the patient's response from the client computer 140 and deliver to the server 110.

After receiving response from the clinic computer 130 or the client computer 140, the server 110 may analyze the information and their corresponding timestamps of the responses. The accumulated timestamps and the corresponding responses can be used to infer the user's preferred communication time, duration, topics, and/or capacity of daily tasks. The daily or weekly tasks may include taking meals (breakfast, lunch, afternoon tea, dinner), exercises, works, and sleep. A collection of tasks in one day constitutes a behavior or a daily schedule of the user. Similarly, a collection of tasks in one week or in one month constitutes a behavior or periodic schedule of the user. In one embodiment, the analysis of the physiological information and corresponding timestamp may be done by inferencing one or more machine learning algorithms/models to obtain a periodic schedule of the user. The periodic schedule is consisted of at least one task.

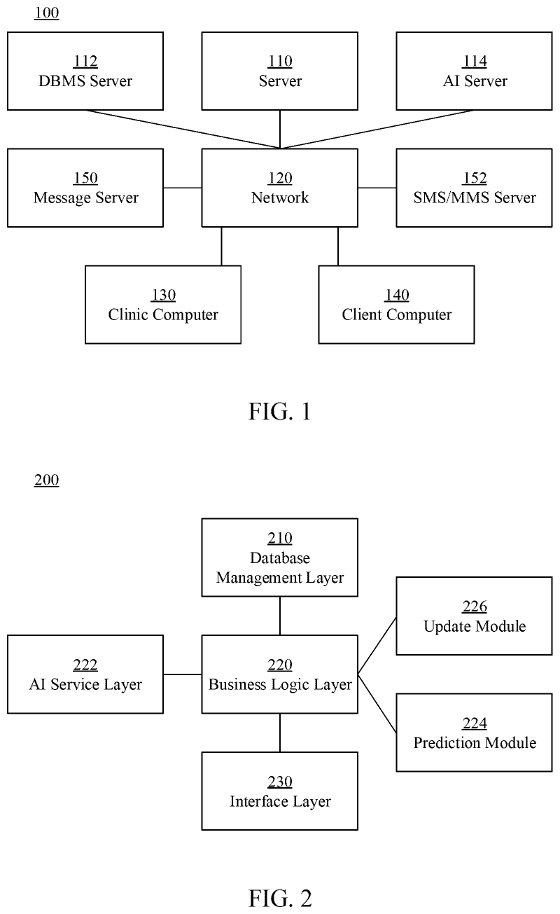

Please refer to FIG. 2, which depicts a software architecture 200 implemented by a server for predicting a communication strategy based on demographic data in a persona database in accordance with an embodiment of the present application. In one example, the software architecture 200 may be solely implemented by the server 110 as shown in FIG. 1. In another example, the software architecture 200 may be collectively implemented by a combination of the server 110, the DBMS server 112, and/or the AI server 114 as shown in FIG. 1.

The software architecture 200 may include a database management layer 210 for operating the persona database, a business logic layer 220 including a prediction module 224 and an update module 226 for implementing the method provided by the present application, an interface layer 230 for interacting with the clinic computer 130 and the client computer 140 via the network 120. Optionally, an AI service layer 222 may be configured to provide inference services for the software architecture 200.

In one embodiment, the database management layer 210 may be installed in the DBMS server 112 as shown in FIG. 1. In one embodiment, the AI service layer 222 may be installed in the AI server 114 as shown in FIG. 1. The DBMS server 112 may include a storage farm for providing large volume of cheap storage space. The AI server 114 may include special hardware such as Graphics Processing Units and/or Neural-network Processing Units for training and/or inferencing the machine learning models provided by the present application. Alternatively, the AI service layer 222 may be provided by commercially available services such as Co-pilot, Gemini, ChatGPT, Perplexity, DeepSeek, and/or any other kinds of AI services.

The server 110 may be capable to implement the machine learning models or Al models by itself. In an alternative embodiment, the AI server 114 may be further utilized by the server 110 to generate the task-specific health specific information of the user. In one example, the AI server 114 may be configured to train or to infer various machine learning algorithms/models which are used in the embodiments of the present application. The machine learning models may be a combination of various neural networks such as convolutional neural networks, deep neural networks, and transformer networks etc. Person having ordinary skill in the art can understand the AI server 114 may be provided by vendors as a cloud service. For examples, Google's cloud service, Amazon's AWS service, and Microsoft's Azure service etc. provide various kinds of neural network services which can be considered as the AI server 114 in the present application. However, without the AI server 114, the server 110 may have enough computing power to implement the machine learning algorithms/models presented in the application.

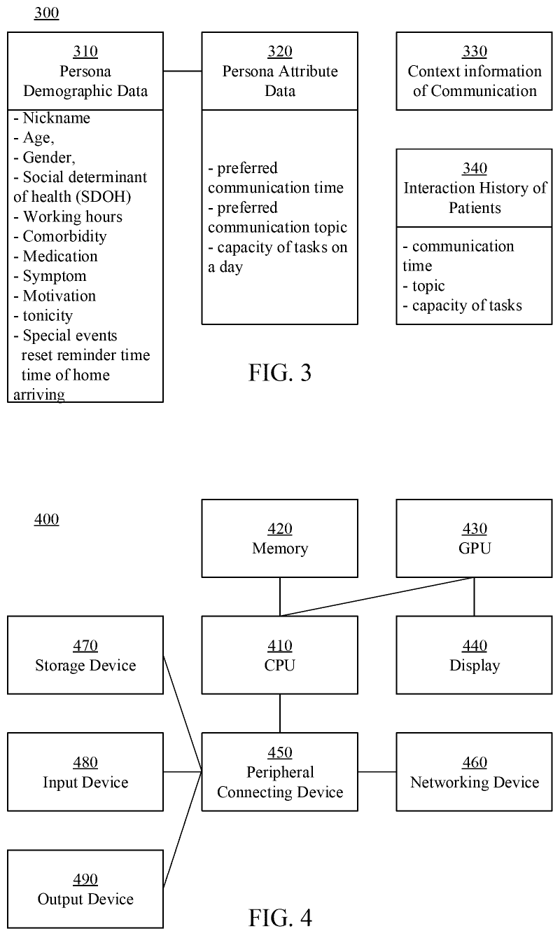

Please refer to FIG. 3, which illustrates a database schema 300 of the proposed persona database in accordance with an embodiment of the present application. Person having ordinary skill in the art can understand that the database schema 300 is suitable for relational databases. However, the database schema 300 may be also varied to be implemented by databases other than relational databases. For the sake of clarity, the database schema 300 as shown in FIG. 3 does not show personal information of patients and/or any other information which may be also included in the persona database.

As described above, with regard to one persona, there are two corresponding data including demographic data 310, attribute data 320, context information of communication 330, and interaction history of patient 340. The demographic data 310 may include one or any combination of age, gender, social determinant of health (SDOH), working hours per week or per month, comorbidity, medication, symptoms, motivation, conversation tonicity, and/or special time event of a patient. The persona attribute data 320 may comprise any combination of following: preferred communication time, preferred communication duration, preferred communication topic, and capacity of tasks in a period (e.g., a day, a week, or a month). Moreover, the demographic data 310 and the attribute data 320 may include more columns which may be mentioned in the present application.

Please refer to FIG. 4, which depicts a block diagram of a computer 400 in accordance with an embodiment of the present application. The various computers depicted in FIG. 1 may be implemented as a variant of the computer 400. Some components of the computer 400 may be absent or altered to fit the role it plays. For example, the AI server 114 may be lack of display 440 and input device 480 such as keyboard and mouse. The GPU (Graphics Processing Unit) 230 of the AI server 114 may be altered to dozens of NPUs (Neural network Processing Units.) Person having ordinary skill in the art may have knowledge of computer organization, computer architecture, system software, and operating system to realize the computers as shown in FIG. 1 based on the block diagram depicted in FIG. 4. Moreover, the clinic computer 130 and the client computer 140 may have input devices 480 such as touch screen, touch panel, keyboard, or buttons to receive the user's or patient's response.

The computer 400 comprises at least one CPU (central processing unit) 410, a memory module 420 for system operation, an optional GPU 430 for generating content to be shown on a display 440, a peripheral connecting device 450 implementing industrial standard interfaces such as PCI, PCI- Express, SCSI, SATA, USB etc., a networking device 460 for connecting to the network 120, a storage device 470 for storing operating system, application programs, and data for implementing the steps provided by the present application, one or more input devices 480, and one or more output devices 490. A operating system run by the CPU 410 is configured to control the computer 400. And various driver programs and application programs under the operating system may be stored in a non-volatile memory such as the storage device 470 and be used to implement the steps of the embodied methods provided by the present application.

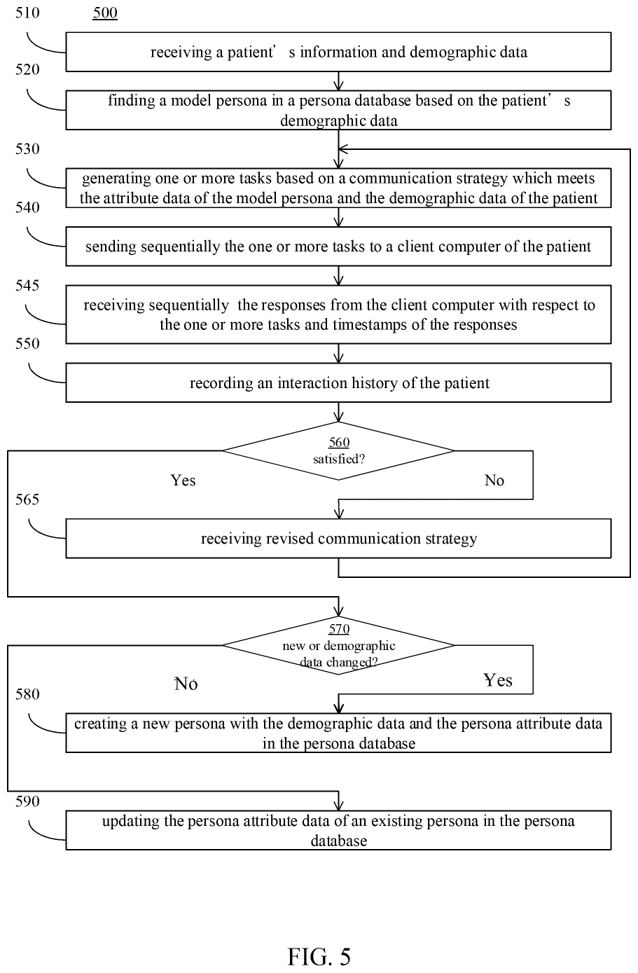

Please refer to FIG. 5, which shows a flowchart diagram of a method 500 for predicting communication strategy based on demographic data in a persona database in accordance with an embodiment of the present application. The method 500 may be implemented by the network system 100, especially by the server 110. If there is no casual relationship directly or indirectly between any two steps as shown in FIG. 5, the present application does not limit their execution sequence. The prediction method 500 may begin at step 510.

Step 510: receiving a patient's information and demographic data. The server 110 may receive the patient's information and demographic data via the clinic computer 130 and/or the client computer 140. In case the patient is new, the flow may proceed to step 520.

Step 520: finding the best fit (either matched or the closest when there is no matched) model persona in a persona database based on the patient's demographic data. Therefore, persona attribute data corresponding to the model persona can be retrieved from the persona database. As discussed above, the best fit model persona may be found in the persona database by a deterministic way or by a machine learning model. Moreover, one or more pre-requisites or constraints may be applied to the finding step.

Step 530: generating one or more tasks based on a communication strategy which meets the persona attribute data corresponding to the model persona and/or the demographic data of the patient.

In one embodiment, the one or more tasks are generated according to context information and a tonicity preference recorded in the demographic data of the model persona. Alternatively, a default tonicity may be employed to generate the one or more tasks. As discussed above, the generating may be helped by one or more LLMs. Each of the tasks is scheduled based on the communication strategy.

In another embodiment, the one or more tasks are generated according to context information and demographic data of the patient. For example, when the patient denotes explicitly time of home arriving is 7:00 pm. Therefore, the communication strategy generated based on the model persona may be altered to have a communication commencing half hour after home arriving, i.e., 7:30 pm. In other words, one or more variables stated in the demographic data of the patient may be taken into account of making communication strategy at this step.

Step 540: sending sequentially the one or more tasks to a client computer of the patient based on the schedules of the one or more tasks. The sending may be directly from the server 110 to the client computer 140. Alternatively, the sending may be indirectly from the server 110 via the message server 150 or the SMS/MMS server 152 to the client computer 140.

Step 545: receiving sequentially the responses from the client computer with respect to the one or more tasks and timestamps of the received responses. In one embodiment, the timestamps are received with the tasks. In an alternative embodiment, the timestamps are generated locally in the server 110.

Step 550: recording an interaction history with the patients based on the steps 540 and 545. In one embodiment, after a round of interactions, the patient may give a satisfactory level with respect to the communication strategy embodied in the round of interactions. The satisfactory level may be a binary value like or dislike. Alternatively, the satisfactory level may be a score from 1to 5 or 1 to 10. A threshold of satisfactory level may be given at 4 in 1 to 5 or at 7 in 1 to 10. In case a value larger than the threshold is evaluated, it means that the patient is satisfied with the communication strategy embodied in the round of communication.

Step 560: determining whether the patient is satisfied with the communication strategy. The determination may be based on the satisfactory score responded by the patient. If the patient is satisfied with the communication strategy embodied in the recorded interaction history at step 550, the flow may end here. Or the flow may further proceed to step 570. Otherwise, If the patient is not satisfied with the communication strategy embodied in the recorded interaction history at step 550, the flow may proceed to step 565.

Step 565: receiving revised communication strategy. In one embodiment, the revised communication strategy may be provided by the patient. In another embodiment, the revised communication strategy may be generated by the server 110. In this embodiment, the revised communication strategy is a variant of the communication strategy used at step 530. After the revised communication strategy is generated by one or a combination of machine learning models, the flow may return to step 530 with the revised communication strategy.

Step 570: Comparing the patient's demographic data to the chosen persona model's demographic data. If there is no difference, the flow proceeds to step 590. Otherwise, the flow proceeds to step 580.

Step 580: If the patient's demographic data are different from the one from the chosen persona model, creating a new persona model with the demographic data and the attribute data mirroring the patient.

Ste 590: updating the persona attribute data corresponding to the patient in the persona database based on the satisfied communication strategy.

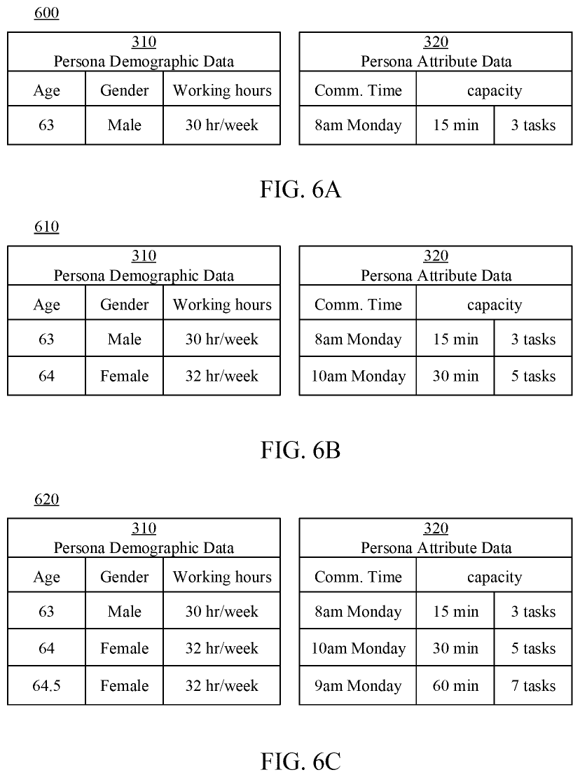

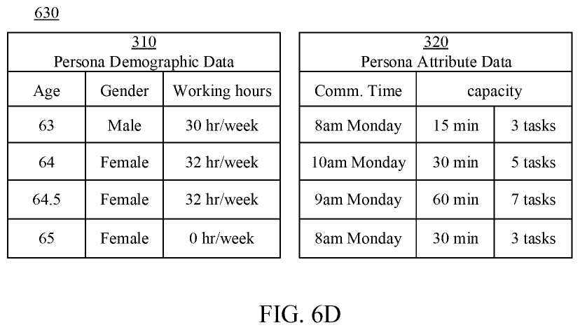

Please find FIGS. 6A˜6D, which depict contents of the persona database in various stages of the method 500 in accordance with an embodiment of the present application. A first table of persona demographic data 310 and a second table of persona attribute data 320 are presented as 600˜630 in FIGS. 6A˜6D, respectively.

In this embodiment, a 64-year-old lady, who is still employed, interacts with the network system 100. At step 510 as shown in FIG. 5, the lady inputs her information and her demographic data into the server 110. Or alternatively, a clinic clerk inputs the lady's information and her demographic data into the server 110. The flow of the method 500 proceeds to step 520.

Assuming at that moment, a model persona in the database is found based on the lady's demographic data. As shown in FIG. 6A, the found model persona closet to the lady is a 64-year-old man who is still working. The model persona has attribute data showing the preferred communication timing for engagement is Monday 8:00 am and the window/capacity of the engagement is about 15 minutes. The man is able to answer 3 questions in the capacity window.

Based on the communication strategy of the model persona attribute data, similar communication strategy is employed for generating three tasks for the lady. At step 540, the prepared three questions are sequentially sent to the client computer of the lady on Monday 8:00 am. And the responses with respect to the three questions are received from the client computer of the lady are received by the server at step 545. Timestamps corresponding to the responses are received with the responses or generated locally in the server. This round of interaction history with the lady is recorded in the database schema 300 as shown in FIG. 3.

However, the lady is not satisfied with the communication strategy. The result of determining at step 560 is not okay. The flow may proceed to step 565 to receive a revised communication strategy. Next week, the flow proceeds to have another round of interaction according to the revised communication strategy. Thus, the steps 530 through 560 repeats again for several iterations.

In one embodiment, at step 565, a latest version of revised communication strategy is inferenced by one or a combination of machine learning models based on the recorded interaction history. By employing the latest version of the revised communication strategy, the step 560 determines that the lady is satisfied. The flow proceeds to step 580 from step 570 because the lady has no record in the persona database. At step 580, a new persona corresponding to the lady is created with her demographic data and her preferred attribute data in the database. The result is shown in FIG. 6B.

Following the embodiment, a half year later, the same lady (64.5-year-old), who is still employed, interacts with the network system 100 again (step 510). At step 520, the model persona in the persona database found is actually herself.

From that persona record found in the persona database as shown in FIG. 6B, the corresponding attribute data of the model persona shows that Monday 10 am is preferred and the window of communication is about 30 minutes. And she can take 5 tasks. Therefore, five tasks are generated at step 530 and scheduled to send one by one on Monday 10 am to her client computer at step 540. At step 545, the responses with regard to these 5 tasks and a satisfactory level are received sequentially by the server 110. This round of interaction history is recorded in the persona database at step 550.

However, at step 560, the lady is not satisfied with this round of interaction. The flow proceeds to step 565 to revise the communication strategy. After several rounds of interaction, the lady finally satisfies with a communication strategy inferenced by one or a combination of machine learning models. Then, the flow proceeds to step 570 from step 560.

At step 560, since the age of the lady is added, it means that the demographic data of the lady is changed. Thus, the flow proceeds to step 580. Please refer to FIG. 6C, new record corresponding to the lady is added with new attribute data. The attribute data shown in the bottom line includes a preferred communication timing on Monday 9 am. And a window of 7 tasks can last one hour.

Again, following the above embodiment, the lady turns 65 is just retired. At step 510, she interacts with the network system 100 again. From the persona database, at step 520, the model persona record which is closest to the lady is still her own record. From this model persona, the communication strategy recorded in the corresponding attribute data includes a preferred communication timing on Monday 9 am. And a window of 7 tasks can last one hour. Therefore, seven tasks are generated according to context information and tonicity preferences and scheduled to send to her client computer on Monday 9 am at step 530.

At step 545, the responses with regard to these 7 tasks and a satisfactory level are received sequentially by the server 110. This round of interaction history is recorded in the persona database at step 550.

However, at step 560, the lady is not satisfied with this round of interaction. The flow proceeds to step 565 to revise the communication strategy. After several rounds of interaction, the lady finally satisfies with a communication strategy inferenced by one or a combination of machine learning models. Then, the flow proceeds to step 570 from step 560.

At step 560, since the age of the lady is added, it means that the demographic data of the lady is changed. Thus, the flow proceeds to step 580. Please refer to FIG. 6D, new record corresponding to the lady is added with new attribute data. The attribute data shown in the bottom line includes a preferred communication timing on Monday 8 am. And a window of 3 tasks can last 30 minutes.

The one or a combination of machine learning models which are employed to inference a revised communication strategy may be trained according to the interaction histories recorded in the persona database. Each round of interaction is labeled with a satisfactory level. A training set including the interaction histories and corresponding labels can be used to train the one or a combination of machine learning models.

In one embodiment, the computation resources required by the training can be provided by the AI server 114 of the network system 100 as shown in FIG. 1. The training can be taken periodically.

According to an embodiment of the present application, a method for predicting a communication strategy with patient is provided. The method for predicting a communication strategy with patient, comprising: receiving a patient's demographic data; finding a model persona in a persona database based on the patient's demographic data; generating one or more first tasks based on a first communication strategy which meets attribute data of the model persona in the persona database; sending sequentially the one or more first tasks based on the first communication strategy to a client computer of the patient via a network; receiving sequentially one or more first responses with respect to the one or more first tasks from the client computer; and recording a first interaction history including the one or more first tasks and the corresponding one or more first responses in the persona database.

Preferably, in order to match the closest record with the patient's demographic data, wherein the demographic data of the model persona is the closest to the demographic data of the patient.

Preferably, in order to add a new record of a new patient, the method further comprises: determining whether the patient is new or the demographic data of the patient is different from the existing demographic data of the patient in the persona database; and creating a new persona record in the persona database with the demographic data of the patient and attribute data corresponding to the first communication strategy.

Preferably, in order to update the record of a previously engaged patient, the method further comprises: when the demographic data of the patient is identical to demographic data of an existing persona record of the patient, updating attribute data of the existing persona record based on the first communication strategy.

Preferably, in order to generate the one or more first tasks based on the tonicity preference of the model persona, wherein the one or more first tasks are generated based on a tonicity preference in the demographic data of the model persona.

Preferably, in order to generate the one or more first tasks based on the tonicity preference of the patient, wherein the one or more first tasks are generated based on a tonicity preference in the demographic data of the patient.

Preferably, in order to generate the one or more first tasks by machine learning models, wherein the one or more first tasks are generated by LLM (large language model).

Preferably, in order to schedule the one or more first tasks based on a special time event like time of home arriving or home leaving of the patient, wherein the one or more first tasks are scheduled according to a special time event in the demographic data of the patient.

Preferably, in order to have another round of interaction when the latest round of interaction is not satisfied, the method further comprises: receiving a first satisfactory level of the first interaction history from the client computer of the patient; generating a second communication strategy when the first satisfactory level of the first interaction history is lower than a threshold; generating one or more second tasks based on the second communication strategy; sending sequentially the one or more second tasks based on the second communication strategy to the client computer via the network; receiving sequentially one or more second responses with respect to the one or more second tasks from the client computer; and recording a second interaction history including the one or more second tasks and the corresponding one or more second responses in the persona database.

Preferably, in order to add a new record of a new patient, the method further comprises: determining whether the patient is new or the demographic data of the patient is different from the existing demographic data of the patient in the persona database; and creating a new persona record in the persona database with the demographic data of the patient and attribute data corresponding to the second communication strategy.

Preferably, in order to update the record of a previously engaged patient, the method further comprises: when the demographic data of the patient is identical to demographic data of an existing persona record of the patient, updating attribute data of the existing persona record based on the second communication strategy.

Preferably, in order to have a third round of interaction when the latest round of interaction is not satisfied, the method further comprises: receiving a second satisfactory level of the second interaction history from the client computer of the patient; generating a third communication strategy when the second satisfactory level of the second interaction history is lower than the threshold; generating one or more third tasks based on the third communication strategy; sending sequentially the one or more third tasks based on the third communication strategy to the client computer via the network; receiving sequentially one or more third responses with respect to the one or more third tasks from the client computer; and recording a third interaction history including the one or more third tasks and the corresponding one or more third responses in the persona database.

Preferably, in order to generate revised communication strategy based on machine learning models, wherein the second communication strategy is inferenced by one or a combination of machine learning models.

Preferably, in order to train the machine learning models, wherein the one or a combination of machine learning models are trained by a training set including interaction histories and labels including satisfactory levels of the corresponding interaction histories.

Preferably, wherein the demographic data of the patient include one or any combination of following: nickname, age, gender, social determinant of health (SDOH), periodic (e.g., daily, weekly, or monthly) working hours, comorbidity, medication, symptoms, motivation, conversation tonicity, and special time event of the patient.

Preferably, wherein the attribute data in the persona database comprises one or any combination of following: preferred communication time, preferred communication duration, preferred communication topic, and capacity of tasks in a period.

Preferably, in order to input the data of the patient with helps of clinic clerk, wherein the demographic data of the patient is received from a clinic computer other than the client computer of the patient.

Preferably, in order to send the one or more first tasks in advance, wherein the one or more first tasks are sent to a message server before their scheduled times, and the message server sends the one or more first tasks according to their schedule times to the client computer, respectively.

Preferably, in order to find the model persona other than a deterministic algorithm, wherein the model persona is found resulted from an inference of a machine learning model with respect to the demographic data of the patient and the persona database.

According to an embodiment of the present application, a server computer, comprising: a networking device configured for connecting with a network; and a processor configured for executing instructions stored in non- volatile memory to realize the abovementioned method for predicting communication strategy with patient.

With the presented method and the server computer, a communication strategy for a new patient or a previously engaged patient with changed demographic data can be suggested quickly by finding a comparable persona in a persona database. And one or a combination of machine learning models may be used to suggest revised communication strategy based on several rounds of interactions between the patient and the network system. The attribute data of patients can be updated to the persona database to broaden the comparison basis. Moreover, interaction histories can be also used as a training set to train the one or a combination of machine learning models to improve the suggestions of communication strategies.

While the invention has been described in terms of what is presently considered to be the most practical and preferred embodiments, it is to be understood that the invention needs not to be limited to the above embodiments. On the contrary, it is intended to cover various modifications and similar arrangements included within the spirit and scope of the appended claims which are to be accorded with the broadest interpretation so as to encompass all such modifications and similar structures.

What is claimed is:

1. A method for predicting a communication strategy with patient, comprising:
receiving a patient's demographic data;
finding a model persona in a persona database based on the patient's demographic data;
generating one or more first tasks based on a first communication strategy which meets attribute data of the model persona in the persona database;
sending sequentially the one or more first tasks based on the first communication strategy to a client computer of the patient via a network;
receiving sequentially one or more first responses with respect to the one or more first tasks from the client computer; and
recording a first interaction history including the one or more first tasks and the corresponding one or more first responses in the persona database.

2. The method of claim 1, wherein the demographic data of the model persona is the closest to the demographic data of the patient.

3. The method of claim 1, further comprises:
determining whether the patient is new or the demographic data of the patient is different from the existing demographic data of the patient in the persona database; and
creating a new persona record in the persona database with the demographic data of the patient and attribute data corresponding to the first communication strategy.

4. The method of claim 3, further comprises: when the demographic data of the patient is identical to demographic data of an existing persona record of the patient, updating attribute data of the existing persona record based on the first communication strategy.

5. The method of claim 1, wherein the one or more first tasks are generated based on a tonicity preference in the demographic data of the model persona.

6. The method of claim 1, wherein the one or more first tasks are generated based on a tonicity preference in the demographic data of the patient.

7. The method of claim 1, wherein the one or more first tasks are generated by LLM (large language model).

8. The method of claim 1, wherein the one or more first tasks are scheduled according to a special time event in the demographic data of the patient.

9. The method of claim 1, further comprises:
receiving a first satisfactory level of the first interaction history from the client computer of the patient;
generating a second communication strategy when the first satisfactory level of the first interaction history is lower than a threshold;
generating one or more second tasks based on the second communication strategy;
sending sequentially the one or more second tasks based on the second communication strategy to the client computer via the network;
receiving sequentially one or more second responses with respect to the one or more second tasks from the client computer; and
recording a second interaction history including the one or more second tasks and the corresponding one or more second responses in the persona database.

10. The method of claim 9, further comprises:
determining whether the patient is new or the demographic data of the patient is different from the existing demographic data of the patient in the persona database; and
creating a new persona record in the persona database with the demographic data of the patient and attribute data corresponding to the second communication strategy.

11. The method of claim 10, further comprises:
when the demographic data of the patient is identical to demographic data of an existing persona record of the patient, updating attribute data of the existing persona record based on the second communication strategy.

12. The method of claim 9, further comprises:
receiving a second satisfactory level of the second interaction history from the client computer of the patient;
generating a third communication strategy when the second satisfactory level of the second interaction history is lower than the threshold;
generating one or more third tasks based on the third communication strategy;
sending sequentially the one or more third tasks based on the third communication strategy to the client computer via the network;
receiving sequentially one or more third responses with respect to the one or more third tasks from the client computer; and
recording a third interaction history including the one or more third tasks and the corresponding one or more third responses in the persona database.

13. The method of claim 9, wherein the second communication strategy is inferenced by one or a combination of machine learning models.

14. The method of claim 13, wherein the one or a combination of machine learning models are trained by a training set including interaction histories and labels including satisfactory levels of the corresponding interaction histories.

15. The method of claim 1, wherein the demographic data of the patient include one or any combination of following: nickname, age, gender, social determinant of health (SDOH), periodic working hours, comorbidity, medication, symptoms, motivation, conversation tonicity, and special time event of the patient.

16. The method of claim 1, wherein the attribute data in the persona database comprises one or any combination of following: preferred communication time, preferred communication duration, preferred communication topic, and capacity of tasks in a period.

17. The method of claim 1, wherein the demographic data of the patient is received from a clinic computer other than the client computer of the patient.

18. The method of claim 1, wherein the one or more first tasks are sent to a message server before their scheduled times, and the message server sends the one or more first tasks according to their schedule times to the client computer, respectively.

19. The method of claim 1, wherein the model persona is found resulted from an inference of a machine learning model with respect to the demographic data of the patient and the persona database.

20. A server computer, comprising: a networking device configured for connecting with a network; and a processor configured for executing instructions stored in non-volatile memory to realize the method as recited in claim 1 for predicting communication strategy with patient.
